(19) United States
(12) Patent Application Publication
(10) Pub. No.: US 2013/0209520 A1
(43) Pub. Date: Aug. 15, 2013

(54) FOAMS, INCLUDING MICROCELLULAR FOAMS, CONTAINING COLLOIDAL PARTICULATES

(71) Applicants: Kosta Ladavac, Somerville, MA (US); Rodrigo Guerra, Cambridge, MA (US); David Kaz, Somerville, MA (US); Vinothan N. Manoharan, Cambridge, MA (US); Jens B. Rieger, Ludwigshafen (DE); Roland S. Koltzenburg, Dannstadt (DE); David A. Weitz, Bolton, MA (US)

(72) Inventors: Kosta Ladavac, Somerville, MA (US); Rodrigo Guerra, Cambridge, MA (US); David Kaz, Somerville, MA (US); Vinothan N. Manoharan, Cambridge, MA (US); Jens B. Rieger, Ludwigshafen (DE); Roland S. Koltzenburg, Dannstadt (DE); David A. Weitz, Bolton, MA (US)

(73) Assignee: BASF SE, Ludwigshafen (DE)

(21) Appl. No.: 13/697,706

(22) PCT Filed: May 20, 2011
(86) PCT No.: PCT/US11/37377

§ 371 Date: Jan. 28, 2013

Related U.S. Application Data
(60) Provisional application No. 61/347,082, filed on May 21, 2010.

(51) Int. Cl. A61K 9/12

(52) U.S. Cl. CPC A61K 9/122
USPC 424/400

(57) ABSTRACT

The present invention generally relates to foams and particles made from such foams, for applications such as drug delivery. The foams or particles may comprise a pharmaceutically acceptable polymeric carrier. In some cases, the foams may include colloidal particulates. A first aspect of the present invention is generally related to polymer-based foams or particles containing pharmaceutically active agents. In some cases, the foam or particle may contain smaller colloidal particulates therein. Such colloidal particulates may be used, for example, to limit the amount of material within certain regions of the foam, or exclude pharmaceutically active agents from being located within certain portions of the foam, which may useful for enhancing release of pharmaceutically active agents from the foam. In some cases, the colloidal particulates may cause the foam or particle to have an unexpectedly high specific surface area. The foam, in certain embodiments, can exhibit a relatively high loading of the pharmaceutically active agent. The foam may be microcellular in certain instances. The foam may also be created using a supercritical fluid, for example, supercritical C0₂. For instance, a precursor to the foam, containing a pharmaceutically active agent, a pharmaceutically acceptable polymeric carrier, and colloidal particulates, can be mixed with a foaming agent. The pressure may then be decreased, thereby causing the foaming agent to expand and causing a foam to form. The foam may also be ground or milled, or otherwise processed, to form particles such as nanoparticles.

### RELATED APPLICATIONS

This application claims the benefit of U.S. Provisional Patent Application Ser. No. 61/347,082, filed May 21, 2010, entitled “Foams Including Microcellular Foams Containing Colloidal Particulates,” by Ladavac, et al., incorporated herein by reference.

### FIELD OF INVENTION

The present invention generally relates to foams and particles made from such foams, for applications such as drug delivery. In some cases, the foams may include colloidal particulates.

### BACKGROUND

Nanoscale particles are of interest to applications such as drug delivery because of their high surface-to-volume ratio. But making nanoscale particles typically involves precipitation and growth. The problem with such methods is that the growth process is difficult to stop, and different precipitation processes are required for different ingredients. Accordingly, improvements in the creation of nanoscale particles are needed.

### SUMMARY OF THE INVENTION

The present invention generally relates to foams and particles made from such foams, for applications such as drug delivery. In some cases, the foams may include colloidal particulates. The subject matter of the present invention involves, in some cases, interrelated products, alternative solutions to a particular problem, and/or a plurality of different uses of one or more systems and/or articles.

In one aspect, the present invention is generally directed to a pharmaceutically active article. In a first set of embodiments, the pharmaceutically active article includes a foam comprising a pharmaceutically active agent, a pharmaceutically acceptable carrier, and colloidal particulates at a density of at least about 1 colloidal particulate/micrometer³ within the foam. The pharmaceutically active article, in another set of embodiments, includes a foam comprising a pharmaceutically active agent and colloidal particulates at a density of at least about 1 colloidal particulate/cell within the foam. In some cases, the pharmaceutically active article also comprises a pharmaceutically acceptable polymeric carrier.

The pharmaceutically active article, in yet another set of embodiments, includes a foam comprising a pharmaceutically acceptable polymeric carrier, a pharmaceutically active agent, and colloidal particulates present within the foam at a concentration of at least about 20% based on the weight of the foam. In still another set of embodiments, the pharmaceutically active article includes a foam comprising a pharmaceutically acceptable polymeric carrier, a pharmaceutically active agent, and colloidal particulates present within the foam at a concentration of at least about 10% based on the volume of the foam.

According to another set of embodiments, the pharmaceutically active article includes a plurality of particles. The plurality of particles may comprise a pharmaceutically acceptable polymer carrier. In certain cases, the particles comprise a pharmaceutically active agent, and the particles may have an average characteristic dimension of no more than about 5 micrometers. In some embodiments, at least about 20% of the discrete particles contain colloidal particulates therein.

In another set of embodiments, the pharmaceutically active article includes a foam comprising a pharmaceutically acceptable polymeric carrier, at least about 20 wt % of a pharmaceutically active agent, and colloidal particulates therein. In some cases, the foam may have an average cell size of less than about 5 micrometers. The pharmaceutically active article, in accordance with another set of embodiments, includes a foam comprising a pharmaceutically acceptable polymeric carrier and colloidal particulates at a density of at least about 1 colloidal particulate/micrometer³ within the foam.

The pharmaceutically active article, in another set of embodiments, includes a plurality of colloidal particulates interconnected by a pharmaceutically acceptable polymeric carrier to form a network having a void fraction of at least about 50 vol %.

In one set of embodiments, the pharmaceutically active article comprises a foam comprising a pharmaceutically acceptable polymeric carrier and colloidal particulates, where (a) the colloidal particulates are present at a density of at least about 1 colloidal particulate/micrometer³ within the foam, (b) the colloidal particulates are present within the foam at a concentration of at least about 20% based on the weight of the foam, (c) the colloidal particulates are present within the foam at a concentration of at least about 10% based on the volume of the foam, and/or (d) the foam has an average cell size of less than about 5 micrometers.

Another aspect of the present invention is generally directed to a method of forming a pharmaceutically active article. In one set of embodiments, the method includes acts of mixing a pharmaceutically acceptable polymeric carrier, a pharmaceutically active agent, and colloidal particulates with a foaming agent to form a precursor of a foam, and subjecting the precursor to a pressure drop whereby the foaming agent expands and forms the pharmaceutically active article as a foam of the precursor. In some embodiments, the density of colloidal particulates in the mixture is at least about 1 particle/micrometers³.

In another aspect, the present invention is directed to a method of making one or more of the embodiments described herein, for example, a polymeric foam such as a microcellular foam or other types of foams or particles as discussed herein containing colloidal particulates. In another aspect, the present invention is directed to a method of using one or more of the embodiments described herein, for example, a polymeric foam such as a microcellular foam or other types of foams or particles as discussed herein containing colloidal particulates.

Other advantages and novel features of the present invention will become apparent from the following detailed description of various non-limiting embodiments of the invention when considered in conjunction with the accompanying figures. In cases where the present specification and a document incorporated by reference include conflicting and/or inconsistent disclosure, the present specification shall control. If two or more documents incorporated by reference include conflicting and/or inconsistent disclosure with respect to each other, then the document having the later effective date shall control.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention. In the figures:

FIGS. 1A-1B show various foam structures and particles in accordance with certain embodiments of the invention;

FIGS. 2A-2B illustrate colloidal particulates used in certain embodiments of the invention;

FIGS. 3A-3D illustrate various polymer foams containing colloidal particulates, in other embodiments of the invention;

FIGS. 4A-4B illustrate grain size distributions of certain foams, in yet another embodiment of the invention;

FIGS. 5A-5B illustrate certain thin film foams, in yet another set of embodiments;

FIGS. 6A-6C illustrate various foams containing particles, in still another set of embodiments;

FIGS. 7A-7C illustrate the effects of wettability and mixing, in certain embodiments of the invention; and

FIG. 8 illustrates dissolution of certain formulations, in yet another set of embodiments.

### DETAILED DESCRIPTION

The present invention generally relates to foams and particles made from such foams, for applications such as drug delivery. The foams or particles may comprise a pharmaceutically acceptable polymeric carrier. In some cases, the foams may include colloidal particulates. A first aspect of the present invention is generally related to polymer-based foams or particles containing pharmaceutically active agents. In some cases, the foam or particle may contain smaller colloidal particulates therein. Such colloidal particulates may be used, for example, to limit the amount of material within certain regions of the foam, or exclude pharmaceutically active agents from being located within certain portions of the foam, which may useful for enhancing release of pharmaceutically active agents from the foam. In some cases, the colloidal particulates may cause the foam or particle to have an unexpectedly high specific surface area. The foam, in certain embodiments, can exhibit a relatively high loading of the pharmaceutically active agent. The foam may be microcellular in certain instances. The foam may also be created using a supercritical fluid, for example, supercritical CO₂. For instance, a precursor to the foam, containing a pharmaceutically active agent, a pharmaceutically acceptable polymeric carrier, and colloidal particulates, can be mixed with a foaming agent. The pressure may then be decreased, thereby causing the foaming agent to expand and causing a foam to form. The foam may also be ground or milled, or otherwise processed, to form particles such as nanoparticles.

In certain aspects, particles such as nanoparticles may be created and controlled by using techniques such as those discussed herein to constrain particle formation. For example, certain embodiments of the invention are generally directed to foams that are created where the material between cells or bubbles within the foam are controlled due to the presence of colloidal particulates within the foam. In such foams, the colloidal particulates may be concentrated in the spaces between cells or bubbles, i.e., in the “plateau regions” or “plateau borders.” The presence of colloidal particulates within these spaces may serve to partially or completely exclude the polymer and/or the pharmaceutically active agent from these locations.

Accordingly, the size of the cells or bubbles and/or the packing density of the foam, in combination with colloidal particulates, may be controlled to control the intercellular spacing within the resulting foam and the size or shape of the particles or nanoparticles that can be created from the foam, e.g., as discussed below. For example, the cells or bubbles within a foam may be controlled to be on the micrometer scale; however, the bubbles may be closely packed together, such that the spaces between them, where the material defining the foam is located, is on the nanoscale. This material can include, for example, a polymer containing a pharmaceutically active agent (i.e., the “active”).

In one set of embodiments, high specific surface areas are achievable by controlling the size and/or packing density of the cells or bubbles, as well as the loading of colloidal particulates within the spaces between the cells or bubbles. Such techniques may be used to create very small domains of active-laden polymer within the foam. The cells or bubbles within the foam may be small (e.g., about 1 micron diameter) and highly packed (e.g., ˜85% volume fraction). The corresponding foam may have borders of a few hundred nanometers, or polymeric foam films below about 50 nm thick. In some cases, colloidal particulates may be present, which may also facilitate high specific surface areas. Such foams may also be processed to form particles or nanoparticles, for example, by grinding or milling the foam, etc.

One aspect of the invention is generally directed to a foam that contains a pharmaceutically active agent, and typically contains colloidal particulates as discussed herein. The foam can have a relatively high specific surface area in certain embodiments such as those discussed below. Such foams can be created using a supercritical fluid, for example, supercritical CO₂. Typically, a foam can include colloidal particulates such as those described herein, a pharmaceutically acceptable polymeric carrier, a pharmaceutically active agent in combination with the carrier, and “cells” or bubbles that are surrounded by the pharmaceutically acceptable polymeric carrier. The cells may also contain a gas, for example, CO₂ or air. FIG. 3 shows non-limiting examples of such foam structures.

As discussed herein, a foam may include a pharmaceutically acceptable polymeric carrier that contains bubbles or “cells.” Such a foam can have an unexpectedly high specific surface area, at least in some cases. The high specific surface area may facilitate delivery or release of a pharmaceutically active agent, at least in certain embodiments. As a specific non-limiting example, a foam may be milled to expose the internal surfaces of the foam, and the resulting milled particles can be administered to a subject.

In comparison with other foams having similar masses, formed using similar techniques (e.g., using supercritical CO₂ as discussed below), and carrying pharmaceutically active agents at relatively high loadings (e.g., at loadings of at least about 5 wt % based on the weight of the foam), foams such as those discussed herein may have much higher specific surface areas than would otherwise be expected for such foams created under such conditions. Accordingly, and without wishing to be bound by any theory, it is believed that such unexpectedly high specific surface areas may be the result of surprisingly high cellular number densities and small cell sizes (e.g., microcellular foams), which can be created by creating well-homogenized precursors and subjecting the precursors to rapid changes in pressure and/or temperature, as is discussed in detail below. For example, the foam may be a “blown foam” in some embodiments. A blown foam is a foam that is formed by mixing or injecting a gas into a liquid, followed by causing the mixture to solidify to form a final foam.

The “specific surface area,” as is used herein is a measure of the total surface area of the foam (both externally and internally, i.e., within the cells) per unit mass of the foam. Typically, the mass of foaming agent within the foam can be neglected relative to the mass of the polymeric carrier, especially if the foaming agent is a gas that is contained or trapped within the foam, and/or if the foaming agent is able to leave the foam after formation, often being replaced by air.

Any suitable technique known to those of ordinary skill in the art can be used to determine the specific surface area. As an example, the specific surface area can be determined using BET, or the specific surface area can be estimated using the average cell size, the volume fraction of the cells, and the density of the polymer forming the foam (see Example 1 for an example). In some embodiments, for instance, if the foam has closed cells, the foam can be ground prior to determining the surface area. The foam can have, for example, a specific surface area of at least about 0.1 m²/g, at least about 0.2 m²/g, at least about 0.3 m²/g, at least about 0.4 m²/g, at least about 0.5 m²/g, at least about 0.6 m²/g, at least about 0.7 m²/g, at least about 0.8 m²/g, at least about 0.9 m²/g, at least about 1 m²/g, at least about 2 m²/g, at least about 3 m²/g, at least about 4 m²/g, at least about 5 m²/g, at least about 6 m²/g, at least about 7 m²/g, at least about 8 m²/g, at least about 9 m²/g, at least about 10 m²/g, at least about 12 m²/g, at least about 15 m²/g, at least about 20 m²/g, at least about 25 m²/g, at least about 30 m²/g, at least about 35 m²/g, at least about 40 m²/g, etc.

The cells may have any shape or size within the foam, and may also have any size distribution. For example, the foam may have an average cell size of less than about 10 micrometers. While cells may vary in shape and/or size, an average cell size can be defined as the average of the characteristic cell size for each cell within the foam, where the characteristic cell size for a cell is the diameter of a perfect sphere having a volume equal to the volume of the cell. Such dimensions are usually estimated, e.g., using SEM (scanning electron microscopy) images, TEM (transmission electron microcopy) images or the like, rather than being precisely calculated, because of the heterogeneous distribution of cell shapes and/or sizes within a typical foam. By examining a suitable number of SEM or TEM images of a foam (for example, that have been chosen from representative locations within the foam), the typical dimensions for the cells within each image may be determined, and then used to determine the average cell size within the foam.

In one set of embodiments, the foam has an average cell size of less than about 5 micrometers. In other embodiments, the foam may have an average cell size of less than about 4 micrometers, less than about 3 micrometers, less than about 2 micrometers, less than about 1 micrometers, less than about 0.5 micrometers, less than about 0.3 micrometers, or less than about 0.1 micrometers. The average cell size may also be greater than about 10 nm, greater than about 100 nm, or greater than about 1 micrometer. In another set of embodiments, the foam has a void fraction of at least about 50 vol %, at least about 60 vol %, at least about 70 vol %, at least about 75 vol %, at least about 80 vol %, at least about 85 vol %, at least about 90 vol %, etc. The void fraction is the volume of cells or bubbles in the foam, compared to the total volume of the foam, i.e., the fraction of the foam that is defined by the cells or bubbles. The void fraction may also be less than about 90 vol %, less than about 70 vol %, or less than about 50 vol %.

One set of embodiments is directed to a “microcellular foam.” Typically, such foams have an average cell size of less than about 100 micrometers, and in some cases, the average cell size may be less than about 10 micrometers, less than about 5 micrometers, less than about 3 micrometers, or less than about 1 micrometer. In other embodiments, the microcellular foam may have an average cell size of between about 0.1 micrometers and about 100 micrometers, or between about 0.1 micrometers and about 10 micrometers.

The number density of the cells that are contained within the foam may also be determined, according to certain embodiments, where the number density of cells in a foam is the number of cells per unit volume. Any suitable technique known to those of ordinary skill in the art can be used to determine or estimate the number density of cells in a foam. For example, SEM or TEM images of a representative number of locations from the foam may be acquired and used to determine or estimate the number density of cells. The foam may have, in some embodiments a number density of cells of at least about 10⁷ cm⁻³, at least about 10⁸ cm⁻³, at least about 10⁹ cm⁻³, at least about 10¹⁰ cm⁻³, or at least about 10¹¹ cm³.

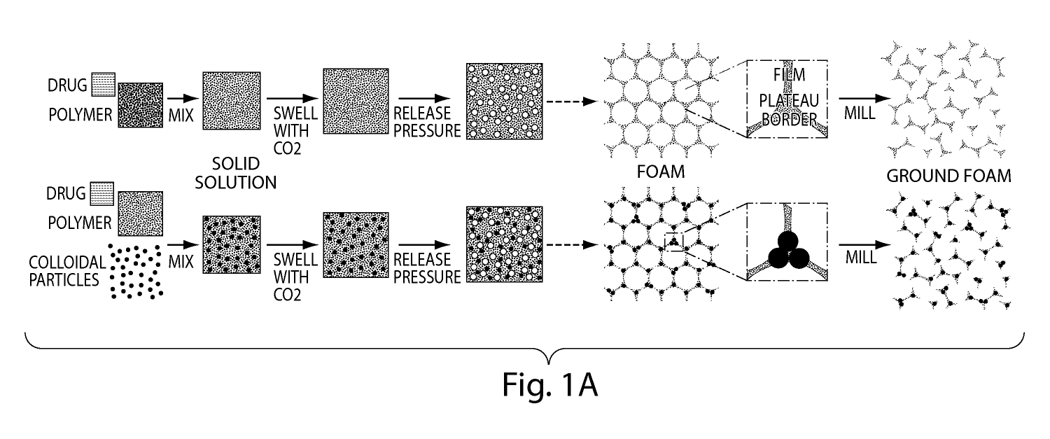

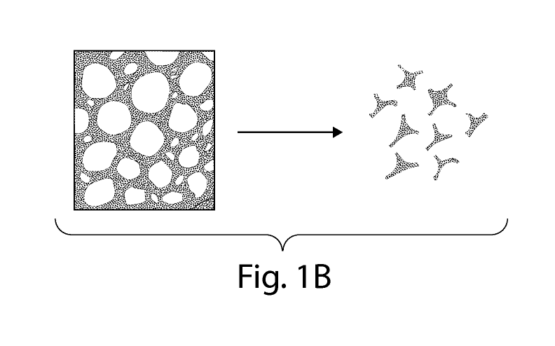

As a non-limiting example, a polymeric carrier may be exposed to a foaming agent that can be dissolved or dispersed within the polymeric carrier at a first temperature or pressure, then by changing the temperature and/or pressure (in some cases, fairly rapidly), the foaming agent may change phase (e.g., forming a gas), which can become trapped within the polymeric carrier, thereby causing bubbles or “cells” to form within the polymeric carrier. This process may be used to create a foam structure in which the polymer forms a matrix surrounding one or more empty regions, or “cells” therein. For example, this can be seen in the schematic diagram of FIG. 1B on the left, where the foam structure includes a number of empty regions or cells therein. The cells may contain a gas, such as CO₂, air, or other foaming agent or the cells may otherwise be substantially free of the polymer.

Non-limiting examples of suitable polymers for use in the pharmaceutically acceptable polymeric carrier include poly(vinyl acetate) or poly(vinylpyrrolidone). Copolymers of these and/or other monomers may also be used in certain embodiments, for example, poly(vinylpyrrolidone-co-vinyl acetate) or polyvinyl alcohol-polyethylene glycol graft copolymer (for example, Kollicoat® IR from BASF). For instance, if a copolymer is used, then the copolymer can be chosen to exhibit any suitable polymeric structure, for example, a block copolymer, a random or statistic copolymer, an alternating copolymer, etc. In certain embodiments, the copolymer may have 2, 3, or more monomers that may be used to define the copolymer. Any suitable ratio of monomers in the copolymer may also be used. For instance, if the copolymer includes vinylpyrrolidone and vinyl acetate, their ratio by weight may be about 6:4, about 4:3, about 1:1, about 2:1, about 3:1, about 10:1, about 1:2, about 1:3, about 1:10, or any other suitable ratio.

The polymer within the pharmaceutically acceptable polymeric carrier can have any suitable molecular weight (also known as molar mass). The molecular weight is often measured as a weight average molecular weight. In some embodiments, the carrier has a molecular weight of at least about 10,000, at least about 20,000, at least about 30,000, at least about 50,000, at least about 70,000, at least about 100,000, at least about 200,000, or at least about 300,000. In some cases, the molecular weight may be no more than 500,000, no more than about 400,000, no more than about 300,000, no more than about 150,000, no more than about 100,000, no more than about 90,000, no more than 80,000, no more than about 70,000, no more than about 60,000, or no more than about 50,000.

The polymer may be chosen, according to certain embodiments of the invention, to have a relatively high affinity for the foaming agent, for example, a relative high affinity for CO₂. As a specific non-limiting example, the foaming agent may be soluble in the polymer at a concentration of at least about 10%, at least about 15%, at least about 20%, at least about 25%, or at least about 30% (determined on a weight basis), at least at operating temperature and pressure. Examples of foaming agents are discussed below in greater detail.

The polymer within the pharmaceutically acceptable polymeric carrier may have, in some embodiments, a relatively low glass transition temperature (T_{g}), i.e., the temperature at which the polymer transitions from a relatively solid state to a more viscous or “rubbery” state, as is known by those of ordinary skill in the art. Any suitable technique known to those of ordinary skill in the art may be used to determine the glass transition temperature for a given material, for instance, by measuring changes in viscosity, using DSC (differential scanning calorimetry), or the like. Typically, the polymer is foamed at a temperature above its glass transition temperature (which varies by material used). However, temperatures that are too high may also be detrimental to some types of pharmaceutically active agents.

Thus, polymers having relatively low glass transition temperatures may be useful, at least in certain embodiments of the present invention. For example, the polymer may be one that exhibits a glass transition temperature of no more than about 200° C., about 180° C., about 160° C., about 150° C., about 140° C., about 130° C., about 120° C., about 110° C., about 100° C., about 90° C., about 80° C., about 70° C., about 60° C., about 50° C., about 40° C., or about 30° C. The glass transition temperature may also be greater than about 0° C., about 10° C., about 20° C., about 30° C., about 40° C., about 50° C., about 60° C., about 70° C., about 80° C., about 90° C., or about 100° C., the foaming agent may be soluble in the polymer at a concentration of at least about 10%, at least about 15%, at least about 20%, at least about 25%, or at least about 30% (determined on a weight basis), at least at Standard Temperature and Pressure (0° C. and 100 kPa or 1 bar). In certain embodiments, the glass transition temperature can be between about 95° C. and about 105° C. In one set of embodiments, the polymer is foamed at a temperature that is relatively close to its glass transition temperature in some embodiments. For instance, the foaming temperature, i.e., the temperature of the polymer when the foaming process is initiated, such as by depressurization of the polymer, may be about 10° C., about 20° C., or about 30° C. above the glass transition temperature of the polymer.

The polymer may also have any suitable material density, according to another set of embodiments. As used herein, the “material density” of a polymer (also known as “bulk density”) is the density of the polymer in the absence of any cells, colloidal particulates, foaming agents, or other non-polymeric materials (such as air or CO₂) that may be trapped within the polymer. However, the “foam density” of a foam can be defined as the overall mass of the foam divided by its volume, which can also include anything trapped within the foam, such as a foaming agent. In certain embodiments, the polymer has a material density of less than about 3 g/cm³, less than about 2 g/cm³, less than about 1.5 g/cm³, less than about 1 g/cm³, less than about 0.8 g/cm³, or less than about 0.5 g/cm³. In some cases, the foam has a foam density of less than about 3 g/cm³, less than about 2 g/cm³, less than about 1.5 g/cm³, less than about 1 g/cm³, less than about 0.8 g/cm³, or less than about 0.5 g/cm³. In addition, it should be understood that the foam density is typically lower than the material density for a given foam.

The polymer within the pharmaceutically acceptable polymeric carrier may be a pharmaceutically acceptable polymer. For instance, the polymer may be bio-inert, biocompatible, or biodegradable. “Biocompatible,” as used herein is given its ordinary meaning in the art. For example, a biocompatible material may be one that is suitable for administration to a subject, without adverse consequences. A pharmaceutically acceptable polymer is thus one that can be swallowed by the subject, and the polymer may be relatively inert and pass through the subject without absorption or adverse consequences, and/or the polymer may be one that is degraded within the subject (i.e., the polymer may be biodegradable), and the products of degradation do not adversely affect the subject. For example, the biodegradable polymer may be one that is water soluble. Examples of biodegradable polymers include, but are not limited to, poly(caprolactone), poly(glycolic acid), poly(lactic acid), poly(3-hydroxybutyrate), etc., as well as copolymers of any of these and/or other suitable monomers. One non-limiting example is poly(lactic acid-co-glycolic acid).

The polymer in the pharmaceutically acceptable polymeric carrier can be selected such that the polymer is water soluble. A water-soluble polymer can exhibit a reasonable rate of dissolution in water, and can be easily screened for and identified. For example, 10 g of the polymer may dissolve within 1 liter of water within less than one week, one day, 12 hours, or 3 hours, etc. Upon administration to a subject, in some embodiments, the polymer begins to dissolve within the subject, thereby releasing at least some of the pharmaceutically active agent internally of the subject. The rate of dissolution of the polymer may be controlled in certain cases, e.g., by adding one or more monomers to the polymer that slow dissolution, and/or by controlling the monomers or the monomer rations within the polymer in order to achieve a desired dissolution speed. For example, the dissolution speed may be increased by copolymerizing a relatively fast-dissolving monomer, such as lactic acid, or the dissolution speed may be decreased by copolymerizing a relatively slow-dissolving monomer, such as glycolic acid.

In addition, in some aspects, the pharmaceutically acceptable polymeric carrier may also contain one or more colloidal particulates. As mentioned, colloidal particulates may be used to limit the amount of material in certain regions within a foam, e.g., by excluding pharmaceutically active agents from being located within certain portions of the foam due to the presence of the colloidal particulates. Typically, little or no penetration into the colloidal particulate by the pharmaceutically active agent or the pharmaceutically acceptable polymeric carrier can occur; thus, the presence of colloidal particulates within the foam structure effectively limits the locations where the pharmaceutically active agent may be present within the foam. In addition, due to the larger size of the colloidal particulates, relative to the pharmaceutically active agent, the colloidal particulates may often exclude the pharmaceutically active agent from being located in positions further away from a surface of the foam.

As used herein, a “colloidal particulate” is a particle, which may be substantially spherical or non-spherical, having a characteristic dimension of no more than about 1 micrometer. In some cases, the characteristic dimension may be no more than about 800 nm, no more than about 600 nm, no more than about 500 nm, no more than about 400 nm, no more than about 300 nm, or no more than about 200 nm. The characteristic dimension of a colloidal particulate is the diameter of a perfect sphere having a volume equal to the volume of the colloidal particulate. Such dimensions can be determined using any suitable technique, e.g., by estimation, e.g., from SEM (scanning electron microscopy) images, TEM (transmission electron microcopy) images or the like, by laser light scattering techniques, or the like. For example, by examining a suitable number of SEM or TEM images to determine typical dimensions for the colloidal particulates within each image, the average characteristic dimension of the colloidal particulates may be determined.

In some embodiments, the colloidal particulates are chosen so as to be bio-inert, biodegradable, or biocompatible. Typically, the colloidal particulates are chosen such that little or no penetration inside the colloidal particulates by the pharmaceutically active agent or the pharmaceutically acceptable polymeric carrier can occur, at least on a time scale of interest. In one set of embodiments, the colloidal particulates may be formed from relatively inert materials, such as silica, for instance, precipitated silica or fumed silica. In another set of embodiments, the colloidal particulates may contain biocompatible or biodegradable polymers, such as poly(lactic acid), poly(glycolic acid), poly(caprolactone), or the like, which in some cases may be formed as a copolymer.

In some cases, relatively high numbers or densities of colloidal particulates may be used in the foam. For example, colloidal particulates may be mixed into the foam such that the density of colloidal particulates appearing within the foam, and/or within particles subsequently formed from the foam, is at least about 1 particulate/micrometer³, at least about 10 particulates/micrometer³, at least about 10² particulates/micrometer³, or at least about 10³ particulates/micrometer³. Such numbers or number densities may, in some cases, be estimated using SEM or TEM images, or other suitable techniques. In another set of embodiments, the density may be at least about 1 particulate/cell, at least about 10 particulates/cell, at least about 10² particulates/cell, or at least about 10³ particulates/cell. In yet another set of embodiments, the density of colloidal particulates may be at least about 10%, at least about 20%, at least about 30%, at least about 40%, at least about 50%, or at least about 60% by weight.

In one set of embodiments, the colloidal particulates may be chosen to be substantially monodisperse. In one set of embodiments, for example, the colloidal particulates have a distribution such that at least about 70% (by number) of the colloidal particulates present have a characteristic dimension that is no more than 10% from an average characteristic dimension of the colloidal particulates. In other embodiments, at least about 80%, at least about 85%, at least about 90%, at least about 95%, or at least about 98% of the colloidal particulates are no more than about 10% from an average characteristic dimension of the colloidal particulates.

As mentioned, the colloidal particulates may be of any shape and/or size. A colloidal particulate may comprise a single particle, or two or more particles, e.g., assembled together as an agglomerate. In some cases, the agglomerate may be formed from smaller subcolloidal particulates, for example, from subcolloidal particulates that have an average characteristic dimension of no more than about 100 nm, no more than about 50 nm, no more than about 40 nm, no more than about 30 nm, no more than about 20 nm, etc. The subcolloidal particulates may be substantially spherical or non-spherical. For instance, in one set of embodiments, the subcolloidal particulates may be sintered or partially melted together to form the final aggregate colloidal particulate. The subcolloidal particulates may be packed at a relatively high density, for example, such that other particles having the same size and/or shape of the subcolloidal particulates will not be able to penetrate the aggregated colloidal particulates.

As mentioned, the pharmaceutically acceptable polymeric carrier forming the foam may also comprise a pharmaceutically active agent. The pharmaceutically active agent may be present within the foam in any suitable amount or concentration, for instance, at a concentration high enough that, when administered to a typical subject, a beneficial or desirable effect is observed. As an example, the pharmaceutically active agent may be present in a foam in an amount of at least about 5 wt % based on the weight of the foam. In some cases, the pharmaceutically active agent may be present at at least about 10 wt %, at least about 15 wt %, at least about 20 wt %, at least about 25 wt %, at least about 30 wt %, at least about 40 wt %, at least about 50 wt %, at least about 60 wt %, or at least about 70 wt % in some cases.

Any suitable pharmaceutically active agent may be used. For example, the pharmaceutically active agent may be one that can be dissolved and/or dispersed within the pharmaceutically acceptable polymeric carrier. For example, a solid solution of a pharmaceutically active agent in a pharmaceutically acceptable polymeric carrier may be formed in some cases, which means that in certain embodiments, the agent may be homogenously distributed within the carrier, although in other embodiments, their distribution need not be homogenous.

In certain embodiments, the pharmaceutically active agent may be one that is not miscible or soluble in water. In some cases, the pharmaceutically active agent is one that is incapable of dissolving in water at ambient temperature and pressure to a concentration of at least 1 g/l. In some cases, however, the pharmaceutically active agent is one that can be homogenously dispersed in water. Examples of pharmaceutically active agents that may be present within the foam include, but are not limited to, carbamazepine, itraconazole, fenofibrate, or clotrimazole.

In one aspect, the foaming agent used to create the foam may be selected to be dissolved or dispersed within a polymeric carrier at a first temperature or pressure to create the foam precursor. The foaming agent may also change phase, for example, into a gas, at a second temperature or pressure that the polymeric carrier is exposed to. In some embodiments, both temperatures and/or pressures may be selected so that the polymeric carrier and/or the pharmaceutically active agent do not substantially degrade. Examples of suitable foaming agents include, but are not limited to carbon dioxide, alkanes such as pentane or hexane, nitrogen, nitrous oxide, or chlorofluorocarbons including hydrochlorofluorocarbons, or mixtures thereof. Other examples include CCl₃F or CCl₂F₂.

By causing the foaming agent to change phase within the precursor, “cells” or pockets may be formed by the foaming agent within the precursor. The cells thereby create the final foam structure of polymer and cells. The foaming agent may be any suitable agent that can be dissolved or dispersed within the polymeric carrier at a first concentration at a first temperature or pressure, but is dissolved or dispersed within the polymeric carrier at a second temperature or pressure at a second concentration that is substantially lower than the first concentration.

The foaming agent may, in some embodiments, change phase between the first temperature or pressure, and the second temperature or pressure. For example, the foaming agent may be dissolved or dispersed in the polymeric carrier at the first temperature or pressure, but forms a gas within the polymeric carrier at a second temperature or pressure. In addition, in some cases, the size of the cells created by the foaming agent in the final foam can be controlled by controlling the homogeneity of the foaming agent within the precursor to the foam, and/or the rate at which the pressure and/or temperature is changed from the first pressure and/or temperature to the second pressure and/or temperature. As one specific non-limiting example, the foaming agent is a gas at Standard Temperature and Pressure (0° C. and 100 kPa or 1 bar).

When mixed with the pharmaceutically acceptable polymeric carrier, the foaming agent can become dissolved or dispersed therein. For example, the foaming agent can be subjected to temperatures and/or pressures such that the foaming agent is not gaseous and can be dissolved or dispersed within the pharmaceutically acceptable polymeric carrier, before foaming, to create a foam precursor, e.g., as discussed below. The precursor can be subjected to a change in pressure and/or temperature that causes the foaming agent, or at least a portion of the foaming agent within the precursor, to form a gaseous state. In one set of embodiments, the change in pressure and/or temperature causes a drop in the amount of foaming agent dissolved or dispersed within the precursor, which then can result in a change of shape, or bubble or cell formation within the precursor.

According to certain embodiments, the foaming agent, when dissolved or dispersed in a pharmaceutically acceptable polymeric carrier to create a foam precursor prior to foaming, can be exposed to pressures and temperatures that cause the foaming agent to be in a supercritical state, wherein the pressure and temperature of the foaming agent, when contacted with the pharmaceutically acceptable polymeric carrier, are each greater than the critical pressure and the critical temperature for that foaming agent. The use of supercritical foaming agents may be advantageous in some instances since a higher concentration of foaming agent may be dissolved and/or dispersed in the pharmaceutically acceptable polymeric carrier, relative to non-supercritical conditions. Accordingly, because of the higher concentration, greater foaming may be produced, e.g., resulting in a higher volume fraction of the cells and/or higher specific surface area of the resulting foam.

A foam may be created by exposing a pharmaceutically acceptable polymeric carrier to a foaming agent to form a precursor, according to another aspect of the invention. The foam may be created using a “batch” process or a continuous process, depending on the application. The pharmaceutically acceptable polymeric carrier may contain a pharmaceutically active agent, and colloidal particulates may also be present in the pharmaceutically acceptable polymeric carrier. The pharmaceutically acceptable polymeric carrier and a pharmaceutically active agent may be mixed together, then the mixture exposed to a foaming agent to form a precursor. The precursor can be exposed to a change in pressure and/or temperature which causes the foaming agent to form a gas, thereby causing the formation of cells within the precursor (containing both the pharmaceutically active agent and the pharmaceutically acceptable polymeric carrier), forming the foam.

In some embodiments, a pharmaceutically acceptable polymeric carrier, colloidal particulates, and a pharmaceutically active agent may be mixed together in any suitable order, for example, simultaneously, sequentially, etc. In some cases, they may be mixed to form a homogenous mixture, for example, a molecular solution of the agent in the polymeric carrier. The pharmaceutically acceptable polymeric carrier and the pharmaceutically active agent may each be in any suitable phase (e.g., solid or liquid), and the mixture may also be, for example, a liquid mixture or a solid mixture.

The pharmaceutically acceptable polymeric carrier and the pharmaceutically active agent may be mixed together directly, or a cosolvent may be used to prepare the mixture. A cosolvent is a material in which the pharmaceutically acceptable polymeric carrier and the pharmaceutically active agent are each mixed with, e.g., dissolved or dispersed, and the cosolvent is then removed, leaving behind a homogenous mixture, such as a solid solution. A cosolvent can be selected such that each of the pharmaceutically acceptable polymeric carrier and the pharmaceutically active agent is able to be dissolved or dispersed within the cosolvent. The specific cosolvent selected may thus be a function of the pharmaceutically acceptable polymeric carrier and the pharmaceutically active agent, and the cosolvent may be water-soluble or water-insoluble, depending on the physical properties of the pharmaceutically acceptable polymeric carrier and the pharmaceutically active agent. For example, if the pharmaceutically acceptable polymeric carrier is poly(vinylpyrrolidone-co-vinyl acetate) and the pharmaceutically active agent is itraconazole, tetrahydrofuran is an example of a cosolvent that can be used. In some cases, the cosolvent may subsequently be removed, e.g., resulting in a powder or a solid which is a homogenous mixture of the pharmaceutically acceptable polymeric carrier and the pharmaceutically active agent. For example, the mixture may be dried or the cosolvent may be partially or completely removed by evaporation and/or heating of the mixture. As another example, the pharmaceutically acceptable polymeric carrier and the pharmaceutically active agent may be mixed together using melt extrusion techniques.

Solid mixtures formed as discussed above may, in some cases, be prepared or processed by milling or grinding the solid mixture to form a powder. For example, techniques such as milling, ball milling, cryomilling, compression, impacting, rollers, crushers, and the like may be used to prepare the solid mixture as a suitable powder. For instance, the solid mixture may be milled using any suitable technique (e.g., ball milling or planetary milling) to form a powder having particle sizes of less than about 1 mm, less than about 500 micrometers, less than about 300 micrometers, less than about 100 micrometers, less than about 50 micrometers, less than about 30 micrometers, less than about 10 micrometers, etc. Smaller particles sizes may be useful, for example, in removing a cosolvent, in promoting more rapid mixing with the foaming agent, etc.

In some cases, the powder may be pressed into pellets or tablets. Such pressing may be useful, e.g., to drive out any gases that may be trapped within the powder matrix, which could adversely affect foaming. Any suitable pressure may be used to press the powder, for example, at least about 1,000 lb/in², at least about 2,000 lb/in², at least about 3,000 lb/in², at least about 4,000 lb/in², at least about 5,000 lb/in², at least about 8,000 lb/in², at least about 10,000 lb/in², etc. (1 lb/in² is about 6.894757 kPa.) Any suitable press, such as a hydraulic press, may be used. The pressure may be applied, in one set of embodiments, until no more creeping is observed in the powder, i.e., such that no more movement or deformation is observed in the powder while pressure is being applied to it. In some cases, an elevated temperature may also be used to facilitate this process, for example, a temperature of at least about 50° C., a temperature of at least about 80° C., a temperature of at least about 100° C., a temperature of at least about 110° C., a temperature of at least about 120° C., etc. For instance, the solid mixture can be exposed to a temperature of between about 90° C. and about 110° C. The solid mixture may be heated before, during, and/or after pressing.

The solid mixture, e.g., formed as a powder or a tablet, etc., can then be exposed to a foaming agent to form a final precursor, which is then processed to form the final foam. In one set of embodiments, the precursor is formed under temperatures and pressures under which the foaming agent is able to be dissolved or dispersed within the solid mixture. For example, the foaming agent may be a gas, a liquid, a solid, or a supercritical fluid. In some cases, after formation of the solid mixture, the solid mixture may be allowed to “soak” the foaming agent into the solid mixture.

As a specific example, in one set of embodiments, the foaming agent is added under conditions in which the foaming agent is supercritical. The exact temperature and pressure used may vary depending on the foaming agent and its critical point. For instance, the temperature at which the foaming agent is added may be at least about 30° C. or at least about 35° C., etc., and/or the pressure at which the foaming agent is added may be at least about 50 atm, at least about 70 atm, at least about 100 atm, at least about 150 atm, at least about 200 atm, at least about 300 atm, at least about 400 atm, at least about 500 atm, etc. As a specific example, the foaming agent may be added at a temperature of between about 30° C. and about 50° C. and a pressure of between about 300 atm and about 500 atm, which are each greater than the supercritical point of CO₂. As another example, the pressure may be between about 350 atm and about 450 atm.

In some cases, the foaming agent may be mixed in the precursor such that the foaming agent forms at least about 5% by weight of the precursor. The foaming agent may also form at least about 10% by weight, at least 15% by weight, at least about 20% by weight, at least about 25% by weight, at least about 30% by weight, at least about 35% by weight, at least about 40% by weight, at least about 45% by weight, at least about 50% by weight, etc., of the precursor.

After formation, the precursor may be caused to form a foam by subjecting the precursor to a change in pressure and/or temperature which causes the foaming agent to form a gas. The exact pressure and/or temperature at which the foaming agent forms a gas may vary depending on the foaming agent. In some embodiments, the precursor may be exposed to ambient (atmospheric) conditions to cause foaming to occur, e.g., about 25° C. and about 1 atm (the actual conditions may vary somewhat). For example, the precursor may be kept in a sealed vessel having a controlled temperature and/or pressure, then the precursor exposed to the ambient environment, e.g., by opening a valve or port in the vessel to the external atmosphere. In other embodiments, the precursor may be exposed to suitable controlled conditions, e.g., having lower temperatures and/or pressures sufficient to cause the foaming agent to form a gas.

In some cases, the decrease in pressure to form a foam may be very rapid. More rapid depressurization rates may affect nucleation rate, which can lead to smaller cells in the final foam. For instance, the change in pressure may occur for a time of less than about 1 s, less than about 500 ms, less than about 250 ms, less than about 200 ms, less than about 150 ms, less than about 100 ms, etc. As a specific example, the change in pressure may occur for a time of between about 100 ms and about 200 ms.

In another aspect, the foam may be ground or milled, or otherwise processed to form particles, including nanoparticles. The particles may have any shape and size, and in some embodiments, these are determined by the initial foam. For instance, a foam containing cells may be broken up to produce discrete particles, where at least a portion of the shape of the particles is determined by the “cells” that were defined in the original foam. Such characteristic shapes may be readily identified by those of ordinary skill in the art, for example, in examining SEM or TEM images.

In some embodiments, the particles may have an average characteristic dimension of less than about 1 mm, and in some cases, less than about 500 micrometers, less than about 300 micrometers, less than about 100 micrometers, less than about 50 micrometers, less than about 30 micrometers, less than about 10 micrometers, less than about 5 micrometers, less than about 3 micrometers, less than about 1 micrometer, less than about 500 nm, less than 400 nm, less than about 300 nm, less than 200 nm, less than 150 nm, less than about 100 nm, less than about 75 nm, or less than about 50 nm in some cases. The “characteristic dimension” of a particle is the diameter of a perfect sphere having the same volume as the particle, and the average of a plurality of particles may be taken as the arithmetic average. In some embodiments, the average characteristic dimension of the particles may be estimated using TEM or SEM images, e.g., of a representative number of particles in a sample.

In certain embodiments, the presence of colloidal particulates within the particles can be determined, e.g., using visualization techniques such as TEM or SEM, or other techniques. For instance, if the colloidal particulates contain fluorescent (e.g., fluorescein) or radioactive materials, the presence of colloidal particulates within the resulting particles can be determined by using fluorescence or radioactivity. In yet another set of embodiments, the colloidal particulates may contain a ferromagnetic material or a magnetically susceptible material that can be determined by determining the magnetic properties of the particles.

Techniques for converting a foam into particles or nanoparticles include, but are not limited to, grinding (e.g., mechanically), milling (e.g., ball milling, planetary milling, cryo-milling), crushing, compression, impacting, rollers, or the like. The duration the technique is applied can also be controlled, e.g., to control the shape and/or size of the particles thereby formed. For instance, longer milling times may result in smaller particles and/or particles having fewer or smaller concave surface regions or portions readily identifiable as cell portions.

In one set of embodiments, the particles have a relatively high surface area. Relatively high surface areas can be achieved in some embodiments since the initial material (e.g., foams) also had a relatively high surface area, and suitable grinding of such foams does not immediately result in perfectly spherical particles, but instead produces irregular forms. For example, the particles so produced may have, in various embodiments, a specific surface area of at least about 0.1 m²/g, at least about 0.2 m²/g, at least about 0.3 m²/g, at least about 0.4 m²/g, at least about 0.5 m²/g, at least about 0.6 m²/g, at least about 0.7 m²/g, at least about 0.8 m²/g, at least about 0.9 m²/g, at least about 1 m²/g, at least about 2 m²/g, at least about 3 m²/g, at least about 4 m²/g, at least about 5 m²/g, at least about 6 m²/g, at least about 7 m²/g, at least about 8 m²/g, at least about 9 m²/g, at least about 10 m²/g, at least about 12 m²/g, at least about 15 m²/g, at least about 20 m²/g, at least about 25 m²/g, at least about 30 m²/g, at least about 35 m²/g, at least about 40 m²/g, etc. In one set of embodiments, particle irregularity may be determined by measuring the average characteristic dimension and the surface area of the particles as a function of mass, and comparing that to the theoretical surface area of spherical particles having the same average characteristic dimension (i.e., diameter) with respect to the same mass basis. The particles of the present invention may have, for example, at least about 1.5 times, at least about 2 times, at least about 2.5 times, at least about 3 times, at least about 4 times, or at least about 5 times the surface area of the theoretical surface area of the spherical particles.

The irregularity or morphology of the particles may be determined using techniques such as electron microscopy (e.g., TEM or SEM). As mentioned, the particles may be created by grinding or milling a foam containing cells into discrete particles containing colloidal particulates, and in some cases, at least a portion of the shape or surface of the particles is determined by the cells that were present in the original foam. In some cases, at least some of the particles will have concave surface regions, as identified using such techniques. Concave surface regions may be created when the materials surrounding or interstitially positioned between the cells or bubbles of the foam are isolated; the isolated solid materials still may retain some of the structure previously defined by the cells or bubbles, thereby retaining a concave surface region in at least one portion of the particle. As an illustration, referring now to FIG. 1B, particles having such shapes may be formed from an initial foam, which is ground to form particles having one, two, or more concave surface regions. In some embodiments, at least about 20%, at least about 30%, at least about 40%, at least about 50%, at least about 60%, at least about 70%, at least about 80%, or at least about 90% of the surface regions of the particle may be defined by one, two, or more concave surface regions.

It should be understood that the particles need not all have the same shape, and in some cases, some of the particles may contain one or more concave surface regions while other particles do not contain readily identifiable concave surface regions, e.g., as can be determined using techniques such as TEM or SEM. However, in the population of particles, at least some of the particles will be identifiable as having one or more concave surface regions. For example, in a sample of particles, on the average, at least about 20% of the particles can be identified as having at least one concave surface region. In some cases, at least about 30%, at least about 40%, at least about 50%, at least about 60%, at least about 70%, at least about 80%, or at least about 90% of the particles may be identified as having one or more concave surface regions.

In some embodiments, at least some of the particles may contain more than one concave surface region. For instance, the particles may be formed at the intersection of two or more bubbles or cells in the original foam. In some cases, at least about 20% of the particles can be identified as having at least two concave surface regions, and in some embodiments, at least about 30%, at least about 40%, at least about 50%, at least about 60%, at least about 70%, at least about 80%, or at least about 90% of the particles may be identified as being “multi-concave,” i.e., having two or more concave surface regions.

The particles may also, in certain embodiments, contain one or more colloidal particulates therein. Such colloidal particulates may be relatively easily identified, for example, using visualization techniques such as TEM or SEM. In some cases, the colloidal particulates may be present within the particles (or within the foam used to produce particles) at concentrations of at least about 1 colloidal particulate/micrometer³, at least about 10 colloidal particulates/micrometer³, at least about 10² colloidal particulates/micrometer³, or at least about 10³ colloidal particulates/micrometer³. The number density of colloidal particulates within the sample may, at least in some cases, be determined using SEM or TEM, or other suitable techniques. In another embodiment, the colloidal particulates may be present within the foam or particles at a concentration of at least about 20% based on the weight of the foam, and in some cases, at least about 25%, at least about 30%, at least about 35%, at least about 40%, at least about 45%, or at least about 50% based on the weight of the foam. In yet another set of embodiments, the colloidal particulates may be present within the foam or particles at a concentration of at least about 20% based on the volume of the foam, and in some cases, at least about 25%, at least about 30%, at least about 35%, at least about 40%, at least about 45%, or at least about 50% based on the volume of the foam.

U.S. Provisional Patent Application Ser. No. 61/160,040, filed Mar. 13, 2009, entitled “Systems and Methods of Templating Using Particles such as Colloidal Particles,” by Weitz, et al.; and PCT Patent Application Serial No. PCT/US2010/000748, entitled “Systems and Methods of Templating Using Particles such as Colloidal Particles,” filed Mar. 12, 2010, by Weitz et al. are each incorporated herein by reference in their entireties.

Also incorporated herein by reference in their entireties are U.S. Provisional Patent Application Ser. No. 61/347,062, filed May 21, 2010, entitled “Foams or Particles For Applications Such as Drug Delivery,” by Ladavac, et al.; U.S. Provisional Patent Application Ser. No. 61/347,082, filed May 21, 2010, entitled “Foams Including Microcellular Foams Containing Colloidal Particulates,” by Ladavac, et al.; and a PCT application filed on even date herewith, entitled “Foams or Particles For Applications Such as Drug Delivery,” by Ladavac, et al.

The following examples are intended to illustrate certain embodiments of the present invention, but do not exemplify the full scope of the invention.

### Example 1

This example illustrates a process for making microcellular polymer foams containing active pharmaceutical ingredients (APIs). These foams can be ground to make small, irregularly shaped particles of API-laden polymer. The large surface area of these foams, and the resulting particles, may increase the dissolution of the API in water, and/or may improve bioavailability. This process disclosed in this example may be well suited-for APIs with low solubility in water. The increase of surface area is due, at least in part, to the foaming step, as the APIs are confined by bubbles or cells. See also a U.S. patent application filed on even date herewith, entitled “Foams or Particles for Applications such as Drug Delivery,” by K. Ladavac, et al., incorporated herein by reference in its entirety. The APIs in this example, however, are confined even further by adding inert colloidal particulates.

The polymer in this example was foamed directly using high pressure supercritical CO₂, eliminating the need for solvents or surfactants. The foam morphology was controlled by the applied pressure, operating temperature, and the pressure release rate. In this example, inert colloidal particulates were used. In the case of foams consisting essentially of polymeric carrier and API, the APIs can be confined by bubbles or cells, which results in increased surface area and the enhanced dissolution of APIs. However, in this example, the APIs were confined both by bubbles and colloidal particulates. For instance, in the foaming step, the total surface area of the resulting foam is governed by bubble size and by bubble volume fraction: smaller bubbles and drier foams (larger volume fraction) are beneficial. However, even for dry foams, most of the material is contained in Plateau borders where the surface-to-volume ratio is lower. Adding colloidal particulates to Plateau borders excludes the API from these low surface area regions, hence increasing the overall surface area and ultimately the solubility. This formulation is general and can be extended to different polymers, APIs, and/or colloidal particulates.

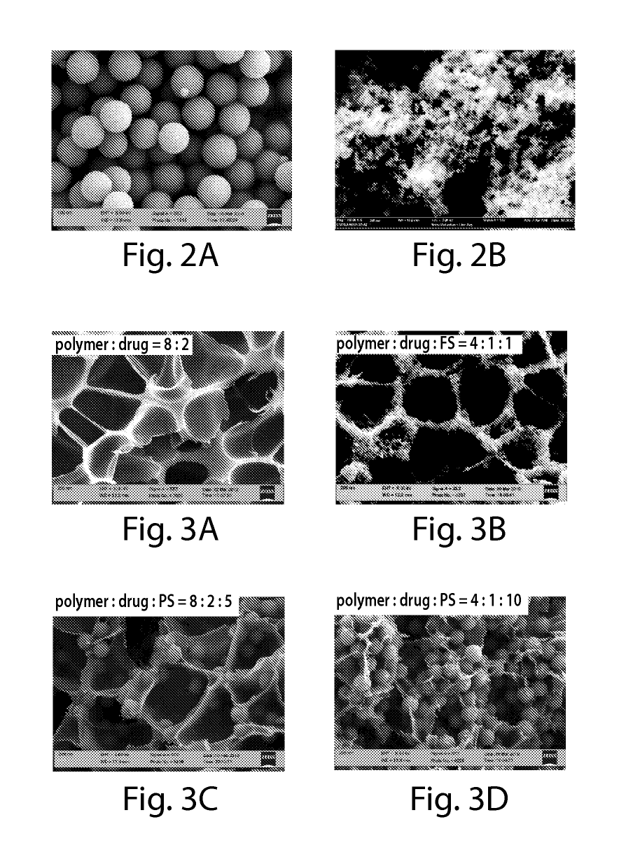

The foam was formed from a polymer, KVA64, poly(1-vinylpyrrolidone-co-vinyl acetate) (Kollidon® VA64, BASF), M_{w}˜45,000-70,000; an API, itraconazole (BASF); and a foaming agent, carbon dioxide (CO₂) (Igo's Welding Supply, Coleman Grade, minimum purity 99.99% liquid phase). Two kinds of particles were used in this example, as depicted in FIG. 2. Both particles were silica, one precipitated and the other fumed. The precipitated silica particles were 380 nm diameter monodisperse spheres (Angstromsphere Monodisperse Silica Powder, Fiber Optic Center Inc., SIO2P025-01). These were full spheres, such that polymer or API could not penetrate inside. In contrast, the fumed silica particles were 200 nm large aggregates of 10 nm to 20 nm spheres sintered together (Cabosil M5, Cabot Corp). These aggregates were fractal, fairly hollow, with a tap density of 0.05 g/cm³ (˜40 times lower than the single particle density). The polymer and API could penetrate inside these aggregates, as these were not efficient confining agents. But they could fill the space at lower loading, and their own surface area was large, as discussed below.

The precipitated silica particles were solid particles. They were not permeable and excluded space from the polymer matrix. At a 33% weight fraction (˜20% by volume) they accumulated in the Plateau borders within the foam, as shown in FIG. 3C. These were the thickest areas, with the lowest surface-to-volume ratio. Since the rest of the foam did not change in the presence of particles (i.e., same bubble size, volume fraction, etc., and therefore the film thickness), this resulted in more surface area available to APIs.

In contrast, precipitated silica loaded at 66% weight fraction (˜50% by volume) appeared to change the foam morphology, as shown in FIG. 3D. The bubbles were smaller, similar to particle size, and each particle appeared to completely fill what would be a Plateau border. The polymer matrix was fully contained in films, now connecting particles instead of connecting Plateau borders. These films were thinner then for any other foam, and the resulting surface area appeared to be larger. In spite of the high particle loading, this may be a feasible approach to increase the surface area available to APIs.

Fumed silica did not appear to change foam morphology, as shown in FIG. 3B. For instance, the bubble or cell size did not appear to change, as compared to foam with no colloidal particulates as is shown in FIG. 3A. Due to the open structure of the fumed particles, their space-filling density was also low. Though these open-structured particles may not have been as efficient in excluding space, as the polymer matrix could permeate inside them, they still may cause the resulting foam structure to be more brittle and easier to break apart. The benefit is that this is achieved at low loading, such that the particles fill the foam's continuous phase, e.g., at 17% by weight.

FIG. 3 shows various foams produced using these techniques, as studied by SEM (scanning electron microscopy). In these figures, PS is precipitated silica, and FS is fumed silica. The polymer used was Kollidon® VA64 and the model drug (API) was itraconazole. The ratios shown here are with respect to weight. The foaming conditions used included CO₂ pressure of 400 atm, 40° C., and a pressure release time of ˜100 ms.

### Example 2

This example illustrates an approach for preparing drug formulations based on confinement rather than synthesis or milling. The premise is that the surface-to-volume ratio of any non-fractal object with smallest dimension h scales as 1/h, regardless of the shape of the object. For example, a large, thin film has a ratio of 2/h, versus 6/h for a sphere with diameter h. Thus it is possible to create high surface areas simply by shrinking the size of the material to the nanoscale in one dimension only.

To implement this idea, inclusions such as bubbles or solid particles were embedded within a solid solution, then packed together to confine the domains of active material into thin films, as shown in FIG. 1A. Nanoscale films were made using relatively large inclusions; the inclusions can be packed together efficiently in some cases, well beyond the 74% volume fraction that can be achieved using close-packed spheres. To this end, bubbles and colloidal particles were used as inclusions in this example. It was found find that the colloidal particles preferentially fill the interstitial regions, called Plateau borders, between the bubbles, changing the structure of the matrix so that it included interconnected thin films having a thickness of 10 nm to 20 nm. As opposed to foams made from the bubbles alone, the particle-packed foams appeared to have no large domains that might dissolve slowly. It was shown that these techniques can increase the dissolution rate of model, hydrophobic actives compared to the state-of-the-art pharmaceutical formulations. The approach is a simple and general route to nanostructured materials. Although shown here with respect to certain pharmaceutical actives, these techniques may be extended to other systems, for example, other actives, polymers, colloidal particles, and the like.

In this example, high pressure CO₂ was used to grow and nucleate micrometer-scale gas bubbles in a solid solution, as shown in FIG. 1A. Certain hydrophobic drugs (“actives”) and a polymer that can dissolve greater than 20% w/w of any of the actives were used in this example. The resulting solid solution was exposed to high-pressure, supercritical CO₂ that swelled the polymer and created free volume between polymer coils, thus depressing the glass transition temperature (T_{g}) of the polymer. Choosing a working temperature between the high-pressure and atmospheric-pressure T_{g} ensured that the pressurized sample was liquid but vitrified when the pressure was released. After a soak time of few hours, the pressure was released, which lead to rapid phase separation and nucleation and growth of CO₂ bubbles. As CO₂ left the polymer, T_{g} increased. When it reached the working temperature, the polymer becomes glassy and the structure was quenched. To make foams with colloidal particles, particles were added as powder when making the solid solution, then the same foaming procedure was followed.

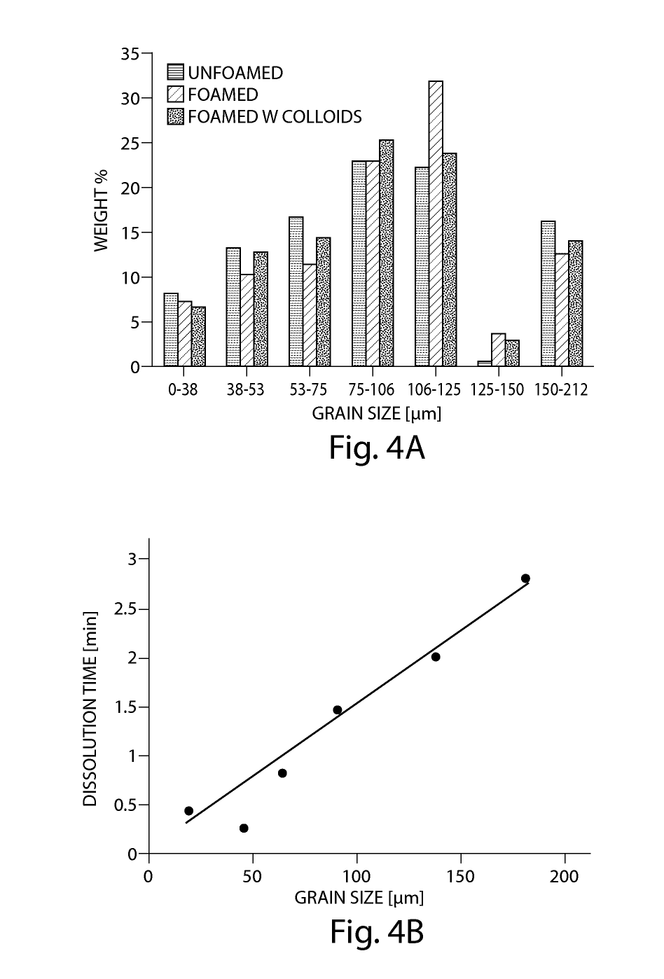

The final step before dissolution was to mill the samples. Milling breaks the bubbles open in the foamed samples so that the interior surface area is accessible to a dissolving liquid. It was found, however, that extended milling may destroy the interior structure of the foam. Because the goal of this example was to investigate the effect of the interior structure on dissolution, and not to create very small particles through milling alone, a gentle cryo-milling technique that broke the samples into large, 10 micrometer to 100 micrometer “chunks” that retained the porous structure of the foam was used in this example. The same milling protocol was used for all of the formulations, including solid solutions, foamed solid solutions, and foamed solid solutions with particles. The resulting grain size distribution of all samples was found to be similar (FIG. 4A). Thus, the grain size distribution appeared to be controlled by milling conditions. This allowed a direct comparison of the dissolution rates of the different formulations.

FIG. 1A shows a schematic diagram of the foam templating process, including polymer matrix, dissolved drug molecules, and colloidal particles. The matrix included a solid solution of drug in polymer, to which colloidal particles were added. The processing steps were the same in both cases. At high pressure, the polymer absorbed CO₂, which precipitated out and nucleated bubbles when the pressure was released. The bubbles grow and pack densely, increasing the surface area. Colloidal particles may pack in the Plateau borders and increase the effective surface area.

To establish a link between surface-to-volume ratio and dissolution rate, dissolution times of milled, sieved solid solutions were determined as a function of grain size. The dissolution time τ (tau) may be defined as the time to dissolve 63% of the active; this definition allows these results to be compared to predictions from the Nernst-Brunner equation, which describes simple diffusion and is often used to model the dissolution rate of pharmaceutical actives:

$$\frac{d C}{d t} = \frac{DA}{Vl} ( {C}_{s} - C ) , \qquad ( 1 )$$

where C is the instantaneous concentration, Cₛ is the saturation concentration (solubility), D is the diffusivity, l is the thickness of the diffusion layer, V is the volume of the medium, and A is the total surface area of dissolving particles. The dissolution time corresponded to the characteristic time τ=Vl/DA in the exponential C˜1−exp(t/τ), which is the solution to Eq. 1. As shown in FIG. 4B, the measured dissolution time τ scales linearly with the grain size, in agreement with Nernst-Brunner.

FIG. 4 illustrates the grain size distribution for foams with clotrimazole, as determined by sieving. FIG. 4A shows the grain size distribution for unfoamed solid solutions (left), foamed solid solutions (center), and foamed solid solutions with colloidal particles (right) after milling. FIG. 4B shows the characteristic dissolution time τ as a function of grain size for unfoamed solid solutions.

In some experiments, it was found that for a given solid solution, the foam morphology was determined by operating pressure, temperature, and pressure release rate. The operating parameters controlled the amount of gas delivered to the polymer, the bubble nucleation rate, and time allowed for bubbles to grow before the polymer vitrifies. Increasing the pressure increased the CO₂ density, yielding more fluid dissolved in the polymer matrix and, in general, a higher final bubble volume fraction and smaller length scales. Decreasing the temperature increased the CO₂ density, leading to more gas dissolved in the polymer, but when the pressure is released there is less time for the bubbles to flow before the structure vitrifies. Decreasing the pressure release time induced a larger thermodynamic instability and a higher oversaturation of CO₂ in the polymer. This lead to a higher nucleation rate, higher volume fraction, and smaller length scales.

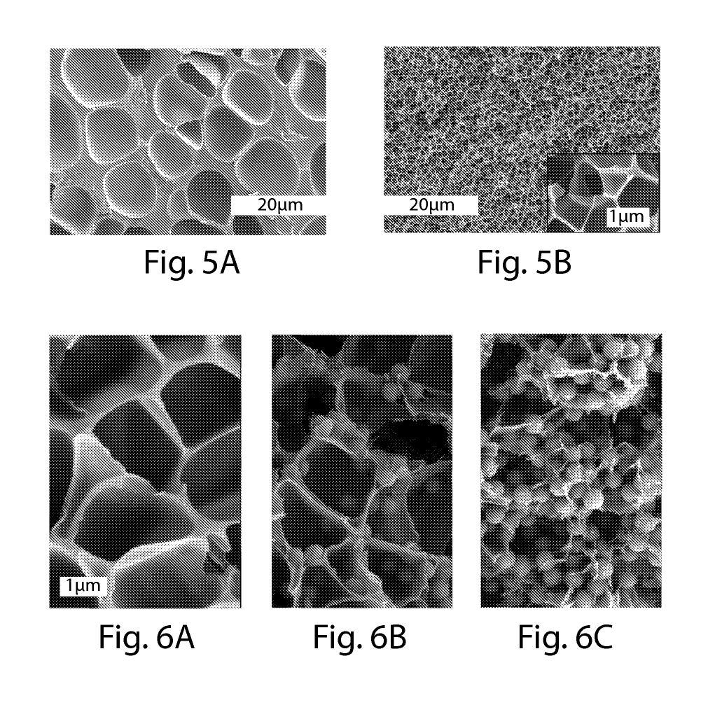

If these parameters are not optimized, the resulting foams may exhibit, in some instances, large bubbles, small bubble volume fractions, thick films, and/or large Plateau borders. For example, foams produced at 100 atm CO₂ pressure with a pressure release time of 3 s exhibited films a few micrometers thick and Plateau borders of approximately 10 micrometers, as shown in FIG. 5A. FIG. 5A is an SEM image of an unoptimized foam made at 100 atm, 50° C., 3 s pressure release time. By increasing the pressure to the maximum allowed by the apparatus used in this example, 400 atm, reducing the pressure release time to ˜200 ms, and optimizing the temperature, foams with micrometer-scale bubbles and volume fractions of 90% were produced. The film thickness was on the order of tens of nanometers, more than an order of magnitude smaller than the bubbles, and the Plateau borders were on the order of hundreds of nanometers, as shown in FIG. 5B, showing an SEM image of an optimized foam made at 400 atm, 40° C., ˜200 ms pressure release time. The inset shows a magnified view of optimized Plateau borders and films.

Although the foaming process leads to a more than 1500% increase in surface area relative to solid solutions (Table 1), some foams were produced that have Plateau borders that are 10 times thicker than the films, as shown in FIG. 6A. The fraction of material contained in Plateau borders was estimated by modeling the Plateau borders as cylinders and the films as sheets. Assuming a close packed polyhedral structure of bubbles, in which each film contacts 5 Plateau borders and each Plateau border connects 3 films, the total volume in the Plateau borders was determined to be twice as large as the total volume contained in films.

TABLE 1

| | | | | foam with | |
|---|---|---|---|---|---|
| | | | foam with | 25% v/v | |
| | | | 25% v/v | hydrophilic | foam with |
| | | | hydro- | particles | 25% v/v |
| | | | philic | poorly | hydrophobic |
| | unfoamed | foam | particles | mixed | particles |
| m²/g | 0.5 ± 0.1 | 8.6 ± 0.2 | 7.7 ± 0.2 | 7.5 ± 0.2 | 9.0 ± 0.2 |
| m²/cm³ | 0.6 ± 0.1 | 10.3 ± 0.3 | 10.4 ± 0.3 | 10.1 ± 0.3 | 12.2 ± 0.3 |

In some experiments, a different type of inclusion, colloidal particles, were used to further reduce the maximum length scales of the material. Before foaming, 380 nm diameter colloidal silica particles were mixed directly with the solid solution. When the silica was homogeneously dispersed in the polymer, homogeneous foams were obtained, as shown in FIG. 6, which shows SEM images of foams prepared using hydrophilic silica particles at 400 atm, 40° C., and ˜200 ms pressure release time. FIG. 6A shows foam with no particles, FIG. 6B shows foam with 25% v/v, and FIG. 6C shows foam with 57% v/v hydrophilic silica particles.

At 25% v/v of particles relative to polymer, the total fraction of inclusions (bubbles and particles) was estimated to be 92%. The bubble size remained the same as when there are no particles, showing that the particles did not significantly influence nucleation of bubbles. Nevertheless, the particles appeared to change the foam morphology: the particles appeared to preferentially accumulate in the Plateau borders, reducing the size of the borders and confining the polymer and active to regions as small as 20 nm.

The reduction in length scale is clearer at higher loading of particles, when the number of particles exceeds the number of Plateau borders that would be present in the pure foam. For instance, at 57% v/v of particles relative to polymer, no empty Plateau borders were observed; also, the bubble size was smaller, on the order of the particle size, and each particle appeared to completely fill what would be a Plateau border (FIG. 6C). The reduction in bubble size was likely related to the decrease in plasticity and reduction of CO₂ solubility of the matrix at these high loadings of particles. The films in these materials were less than 10 nm thick. However, there may be a reduced loading of the active ingredient, since the particles did not contain the active. The foams with 25% particles showed a particle loading that balances the increase in surface area with the decrease in active loading.

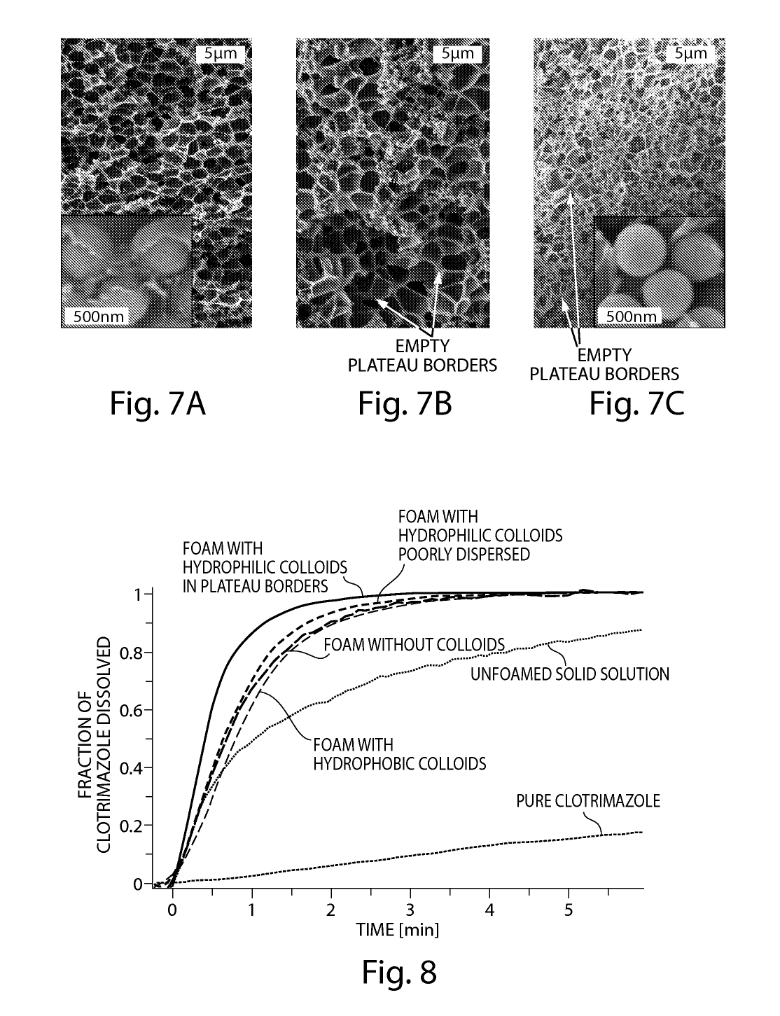

To investigate the effect of the wettability of the particles on the foam structure, the silica particles were functionalized in some experiments with a long alkyl chain to make them hydrophobic, then added to the polymer matrix before foaming. Because the polymer is water-soluble, the polymer was expected to wet hydrophilic silica better than CO₂. Indeed, unmodified silica particles remained engulfed by the polymer and ended up in the Plateau borders, covered with thin films of polymer, as seen in FIG. 7A. By contrast, hydrophobic particles protruded from the polymer (FIG. 7C), although the bubble size remained similar to that of foams made with hydrophilic particles.

FIG. 7 shows SEM images of foams prepared at 400 atm, 40° C., ˜200 ms pressure release time. All samples contained 25% v/v colloidal particles in polymer. FIG. 7A shows foams with well-dispersed hydrophilic particles: particles ended up in Plateau borders. FIG. 7B shows foams with poorly-dispersed hydrophilic particles: particles left many Plateau borders unfilled. FIG. 7C shows foams with hydrophobic particles: many Plateau borders were unfilled, and particles protruded from the polymer. Insets show magnified views of polymer wetting (FIG. 7A) or expelling particles (FIG. 7C).

Foams with hydrophilic particles had a lower surface-to-mass ratio than foamed solid solutions (Table 1), as measured through nitrogen adsorption after milling. The main effect of adding hydrophilic particles appeared to be to increase the average mass density of the foam, since silica was denser than the polymer. Correcting for this effect yielded surface-to-volume ratios that were substantially the same, within measurement uncertainties, as those for pure foams. Hydrophobic particles increased the specific surface area by 20%, even after taking into account the increase in mass density. These results were consistent with observations from electron microscopy showing that the hydrophilic particles were engulfed by polymer and thus did not contribute to the surface area, whereas the hydrophobic particles protruded into the pores.

Changing the surface functionality of the particles also affected how they were dispersed in the polymer matrix. In the sample with 25% v/v hydrophilic particles nearly all Plateau borders were filled with particles (FIG. 7A), but in a foam with the same volume fraction of hydrophobic particles (FIG. 7B), there were many Plateau borders without any particles inside. The reason appeared to be that the hydrophobic particles dispersed poorly in the polymer, and occulated before foaming. Thus there were two effects of changing the surface functionality of the particles: particles protruded from the Plateau borders because of the reduced wettability, and the fraction of filled Plateau borders decreased because of occulation of the particles. To determine how these two effects might affect the surface-to-volume ratio and the dissolution behavior, a control sample was prepared containing hydrophilic particles that were deliberately mixed poorly into the polymer. This inhomogeneous dispersion of particles yielded an inhomogeneous foam. The bubble size was found to be similar to the well-mixed dispersion, but aggregates of particles remained, and the particles filled only a small portion of Plateau borders, as shown in FIG. 7B. The surface-to-volume ratio of this sample was also comparable to that of the foam with well-mixed hydrophilic particles, as expected.

How do the structural changes caused by particles affect how these foams function as drug delivery vehicles? Their functionality was assessed by measuring the dissolution rate in a mixing chamber in the presence of standard pharmaceutical excipients. The in vitro dissolution rate was correlated to the in vivo bioavailability of the drug. The dissolution behavior of all of the foam structures was determined as well as of the pure drug and the unfoamed solid solution, which is the current, state-of-the-art general formulation for oral drug delivery.

All of the formulations, containing model actives, dissolved faster than powders of the pure crystalline drug. The foamed samples, in general, dissolved faster than the unfoamed solid solutions, although in some cases the difference was difficult to resolve because the dissolution times approached the residence time of the experimental apparatus. To better quantify the initial dissolution rate, which would be expected to be proportional to the surface-to-volume ratio, a slowly dissolving drug, clotrimazole, was used.

It was also found that foams with well-dispersed hydrophilic particles dissolved nearly 4 times faster than unfoamed solid solutions and nearly twice as fast as foamed solid solutions, as shown in FIG. 8. Foams with hydrophobic particles or poorly-dispersed hydrophilic particles dissolve at roughly the same rate as foams with no particles. FIG. 8 shows dissolution tests for formulations with clotrimazole in 10 mM SDS solution. The base solid solution is 20% clotrimazole in PVPVA. Dissolution profiles for pure clotrimazole, unfoamed solid solution, foamed solid solution without particles, foamed solid solution with 25% v/v hydrophobic particles (dashed), foamed solid solution with 25% v/v hydrophilic particles, and foamed solid solution with 25% v/v poorly-dispersed hydrophilic particles (dashed) are shown. The initial dissolution rate of foamed solid solutions with hydrophilic particles was 1.8 times faster than the foamed sample without particles, and 3.8 times faster than the unfoamed solid solution.

These results, taken together with the surface area measurements and electron micrographs, showed that the increase in dissolution rate was apparently related to how particles change the structure of the Plateau borders in the foam. Well-mixed, hydrophilic particles filled the majority of the Plateau borders, reducing the amount of material in the largest and slowest-to-dissolve domains and increasing the effective surface-to-volume ratio. The increase in surface area cannot be directly measured by nitrogen adsorption because the particles are still in contact with the films, but indirect evidence comes from dissolution tests of foams with hydrophobic or poorly-dispersed hydrophilic particles. These foams differed in their specific surface areas and in the structure of the Plateau borders, but they shared a common feature: many of the Plateau borders are not occupied by particles. The similarity between dissolution behavior of these foams and of foams without particles suggested that the filling of the Plateau borders was more important determinant of functionality; wetting was relatively less relevant.

Thus, the effect of adding particles to foams appeared to be primarily geometrical. The combination of bubbles and colloidal particles packed more efficiently than either type of inclusion alone, confining the matrix material to nanoscale films throughout the material. This structural effect could be achieved through a simple, scalable physical process that enhances the functionality of the state-of-the-art chemical formulation, the solid solution.

Although the confinement process demonstrated in this example was suitable for actives with low solubility in water, it is general and can be extended to other polymers, actives, and colloids. Inert colloidal particles were used here, but in general, the particles could also play a functional role. For example, the particles could be used to deliver more gas to the polymer matrix, and/or yield finer foam features. The particles might also be used as a reservoir for active materials, or other materials.

All materials were used in these experiments as received, including PVPVA, poly(1-vinylpyrrolidone-co-vinyl acetate) 6:4, (Kollidon VA 64, BASF, CAS 25086-89-9); CO₂, carbon dioxide (Coleman Grade-Min. Purity 99.99% Liquid Phase); 380 nm silica particles (Angstromsphere Monodisperse Silica Powder, Fiber Optic Center Inc., SIO2P025-01); Clotrimazole (Selectchemie, Lot No. 20051116); itaconazole (Selectchemie, Batch No. IT0070709); fenofibrate (Aldrich, Batch No. 017K1401); carbamazepine (Pfannenschmitt, Batch No. 07092639); cholesterol (Alfa Aesar, 96% pure, CAS 57-88-5).

Hydrophobic silica preparation. To make hydrophobic particles, 30 g hydrophilic silica particles were suspended in 30 ml ethanol, and 90 g of long chain alcohol (eicosanol, C20H41OH) were added. The mixture was heated to 90° C. under vacuum to remove ethanol, then further heated to 180° C. and kept overnight while stiffing and under nitrogen. After cooling, the particles were cleaned by washing 5 times in a chloroform/hexane mixture (1:1 by volume) and then dried under reduced pressure.

Preparing solid solutions. Two methods to prepare solid solutions of active in polymer. When small amounts were needed, a co-solvent method was used, in which active and polymer were dissolved in a common solvent, either acetone or ethanol, which was then removed by evaporation. To accelerate drying, the solution was spread on a sheet, dried overnight at 50° C., milled, and dried again. The powders were then heated to a molten state at 120° C. and pressed (Carver 24-ton hydraulic press) to make the final bulk pellets. Because small amounts of residual solvent may substantially reduce T_{g}, the solid solutions were analyzed with a Thermogravimetric Analyzer (TGA, TA Instruments Q5000IR) and a Differential Scanning calorimeter (DSC, TA Instruments Q200). Fully dried samples showed no significant loss of weight through heating, indicating no residual solvent, and a single glass transition temperature, a signature of true solid solution.

For larger amounts of sample (˜10 g), hot melt extrusion was used. Polymer and drug were directly mixed in a small-scale twin-screw extruder (Micro Compounder, DACA Instruments). To ensure full dissolution of the drug in polymer, the extrusion was performed above the melting point of the drug. For example, an operating temperature of 160° C. for clotrimazole was used. High performance liquid chromatography (Agilent 1100 HPLC) showed that the drug did not degrade during heating.

To prepare foams with colloidal particles, silica powder was mixed into the polymer matrix. To make a well-mixed suspension, powders of polymer, active, and particles were combined, and then extruded as described above. To make a poorly-mixed sample, a solid solution was first milled, then the resulting powder was combined with silica particles, mixed gently, and then melted to make the final bulk pellets.

Foaming. To foam the solid solutions, a custom-built apparatus was prepared, including a CO₂ cylinder, pump, and chamber. Gas was drawn from the cylinder to a high pressure syringe pump (model 260D, Teledyne Isco, Lincoln Nebr.) connected to a 100 ml hand-tight steel chamber (made by Pressure Products Industries Inc., Warminster, Pa., purchased from Supercritical Fluid Technologies Inc., Newark, Del.). The pressure was set by the pump, and the temperature was set by a heating sheet wrapped around the chamber. The heating sheet was powered through a PID controller (Omega Engineering, CSI32K iSeries Benchtop controller) that maintains the working temperature with a feedback loop through a thermocouple (Omega Engineering, KHSS-18G-RSC) mounted in the chamber. The pressure release times were made as small as possible by reducing the amount of dead volume in the chamber and quickly venting the CO₂ through a pneumatically activated 3-way valve (Swagelok, SS-H83XPF2-53S). This experimental apparatus handled up to 500 atm pressure, 200° C. temperature, and pressure release times as short as 100 ms. In a typical experiment, 1 gram of solid solution was added to the chamber, allowed to soak for 4 hours at 40° C. and 400 atm pressure, and then pressure is released within 200 ms.

Choice of polymer. The polymer to be used for the solid solution was selected in this example to absorb enough CO₂ at reasonable pressures to make a foam with high volume fraction, and its T_{g} must allow a working temperature that is low enough for this apparatus, but above 31.1° C., to ensure the CO₂ was supercritical. For this particular example application, oral drug delivery, the polymer also needed to be water soluble and approved for ingestion.

In these experiments, PVPVA, a random copolymer of PVP (poly(vinylpyrrolidone)) and PVAc (poly(vinyl acetate)), was selected. PVAc provided high affinity for CO₂ (absorbs more than 20% w/w at 25° C.), while PVP made the whole copolymer more water soluble and is itself a good solvent for many drugs. The glass transition temperature of the pure copolymer was found to be 108° C., above the critical point of CO₂, but well within the working temperature of this experimental apparatus. Adding actives to the polymer typically reduced the T_{g} of the polymer; this was compensated for by adjusting the temperature of the foaming process.

Imaging. A Zeiss Ultra/Supra scanning electron microscope was used to image the foam samples. To expose the structure for imaging without damaging it, the foam was frozen in liquid nitrogen to make the polymer brittle and carefully fractured with a sharp blade. The polymer was nonconducting, so to reduce charging under the electron beam, a thin layer of platinum/palladium was sputtered there.

Milling and characterizing. The samples were loaded in a 50 ml stainless steel jar with one 25 mm stainless steel ball and milled for 2 minutes at 10 Hz while the jar flushed with liquid nitrogen (CryoMill, Retsch Corp.) The surface area of the milled samples was measured by nitrogen adsorption through the BET method (Beckman Coulter Surface Area Analyzer SA3100). To measure the size distribution of milled powders, the powders were first separated by grain size using a Cole Parmer Sieve Shaker, vibrating at 60 Hz, 1 s tapping, with a stack of stainless steel sieves (ASTM E-11 standard) decreasing in mesh size from top to bottom. After sieving for 20 minutes the final contents of each sieve were weighed to determine the grain size distribution.

Dissolution tests. To measure dissolution rates, a custom-built apparatus was used that included a dissolution chamber, a peristaltic pump, and a UV-VIS spectrophotometer. The dissolution chamber (Millipore Solvent-Resistant Stirred Cell 76 mm) was connected through a peristaltic pump to a quartz flow cell (Starna Cells) mounted in the UV-VIS spectrophotometer (Perkin Elmer Lambda 40). The bottom of the chamber was fitted with a filter membrane (Sterlitech PTFE laminated membrane, either 0.2 or 0.45 micrometer pore size) that prevented any undissolved particles from reaching the spectrophotometer. A magnetic stirring bar was mounted in the dissolution chamber and actuated by a magnetic stir-plate below the chamber. The solution flowed through continuously, with the spectrometer measuring the absorbance of the dissolved drug. To relate the absorbance to concentration, samples of known concentration were measured in a good solvent such as ethanol.

After reaching the spectrometer cell, the solution was recirculated back to the chamber, keeping the total volume constant. The total delay time between the addition of the sample to the chamber and the appearance of a steady signal on the spectrophotometer was approximately 30 seconds. This set the time resolution of the measurement.

Samples were applied as powder directly into the dissolution chamber. To help the powder sink and prevent clumping, the powder was mixed with a spacer, an inert material that does not interact chemically with either the active or the polymer. Either microcrystalline cellulose (20 micrometer powder, Aldrich) or fumed silica (sintered aggregates 200 nm large, composed of 10 nm particles, CAB-O-SIL M5, Cabot Corp.) was used. The choice of spacer did not affect the measured dissolution rate.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. A pharmaceutically active article, comprising:
a foam comprising a pharmaceutically acceptable polymeric carrier and colloidal particulates, wherein (a) the colloidal particulates are present at a density of at least about 1 colloidal particulate/micrometer³ within the foam, (b) the colloidal particulates are present within the foam at a concentration of at least about 20% based on the weight of the foam, (c) the colloidal particulates are present within the foam at a concentration of at least about 10% based on the volume of the foam, and/or (d) the foam has an average cell size of less than about 5 micrometers.

2-11. (canceled)

12. The pharmaceutically active article of claim 1, wherein the pharmaceutically acceptable polymeric carrier exhibits a glass transition temperature of at least about 90° C.

13-14. (canceled)

15. The pharmaceutically active article of claim 1, wherein the pharmaceutically acceptable polymeric carrier comprises poly(vinylpyrrolidone).

16. The pharmaceutically active article of claim 1, wherein the pharmaceutically acceptable polymeric carrier comprises poly(vinyl acetate).

17-21. (canceled)

22. The pharmaceutically active article of claim 1, wherein at least some of the colloidal particulates comprise silica.

23-24. (canceled)

25. The pharmaceutically active article of claim 1, wherein the colloidal particulates have an average characteristic dimension of no more than about 1 micrometer.

26-27. (canceled)

28. The pharmaceutically active article of claim 1, wherein at least about 90% of the colloidal particulates by number have a characteristic dimension that is no more than about 10% from an average characteristic dimension of the colloidal particulates.

29-30. (canceled)

31. The pharmaceutically active article of claim 1, wherein at least some of the colloidal particulates are aggregates of subcolloidal particulates.

32-38. (canceled)

39. The pharmaceutically active article of claim 1, wherein the foam has a specific surface area of at least about 0.4 m²/g.

40-51. (canceled)

52. The pharmaceutically active article of claim 1, wherein the foam has a cellular number density of at least about 10⁸ cm⁻³.

53-56. (canceled)

57. The pharmaceutically active article of claim 1, wherein the foam has a void fraction of at least about 50 vol %.

58-63. (canceled)

64. A pharmaceutically active article, comprising:
a plurality of particles, the particles comprising a pharmaceutically active agent and a pharmaceutically acceptable polymeric carrier, and having an average characteristic dimension of no more than about 5 micrometers, wherein at least about 20% of the discrete particles contain colloidal particulates therein.

65. The pharmaceutically active article of claim 64, wherein at least some of the colloidal particulates comprise silica.

66-67. (canceled)

68. The pharmaceutically active article of claim 64, wherein at least some of the colloidal particulates are aggregates of subcolloidal particulates.

69. (canceled)

70. The pharmaceutically active article of claim 68, wherein at least some of the subcolloidal particulates are substantially spherical.

71. (canceled)

72. The pharmaceutically active article of claim 64, wherein at least about 20% of the particles have at least two concave surface regions.

73-76. (canceled)

77. The pharmaceutically active article of claim 64, wherein the plurality of particles has a specific surface area of at least about 6 m²/g.

78-88. (canceled)

89. A method of forming a pharmaceutically active article, comprising:
mixing a pharmaceutically acceptable polymeric carrier, a pharmaceutically active agent, and colloidal particulates with a foaming agent to form a precursor of a foam, wherein the density of colloidal particulates in the mixture is at least about 1 particle/micrometers³; and
subjecting the precursor to a pressure drop whereby the foaming agent expands and forms the pharmaceutically active article as a foam of the precursor.

90. (canceled)

91. The method of claim 89, wherein the foam is microcellular.

92. The method of claim 89, wherein the foaming agent comprises CO₂.

93. The method of claim 89, wherein the foaming agent is mixed with the pharmaceutically acceptable polymeric carrier under conditions such that the foaming agent is supercritical.

94. The method of claim 89, comprising mixing the pharmaceutically acceptable polymeric carrier and the foaming agent at a temperature of at least about 30° C.

95-96. (canceled)

97. The method of claim 89, comprising mixing the pharmaceutically acceptable polymeric carrier and the foaming agent at a pressure of at least about 50 atm.

98-105. (canceled)

106. The method of claim 89, wherein the pressure drop is applied for a time of less than about 1 s.

107-109. (canceled)

110. The method of claim 89, wherein the precursor is formed by mixing the pharmaceutically acceptable polymeric carrier and a pharmaceutically active agent with a cosolvent, and removing the cosolvent.

111-116. (canceled)

117. The method of claim 89, wherein the precursor is exposed to a pressure of at least about 5,000 lb/in².

118. (canceled)

119. The method of claim 89, wherein the precursor is exposed to a temperature of at least about 80° C.

120-122. (canceled)

123. The method of claim 89, further comprising processing the foam to form particles.

124-139. (canceled)
